(19) United States
(12) Patent Application Publication
(10) Pub. No.: US 2025/0312193 A1
(43) Pub. Date: Oct. 9, 2025

(54) INTRAOCULAR PRESSURE DEVICES

(71) Applicant: Alcon Inc., Fribourg (CH)

(72) Inventors: Razek G. Coussa, Oklahoma City, OK (US); Reto Grüebler, Greifensee (CH); Christoph Siegenthaler, Eschenz (CH)

(21) Appl. No.: 19/068,233

(22) Filed: Mar. 3, 2025

Related U.S. Application Data
(60) Provisional application No. 63/573,789, filed on Apr. 3, 2024.

(51) Int. Cl. A61F 9/007

(52) U.S. Cl. CPC A61F 9/00781; A61F 2220/0025

(57) ABSTRACT

An intraocular pressure (IOP) maintenance device for maintaining IOP is disclosed. The IOP maintenance device includes an inlet configured to receive fluids from a first portion of the eye, an outlet configured to drain the fluids into a second portion of the eye, and a pressure regulator disposed between the inlet and the outlet, the pressure regulator comprising a pressure resistance apparatus configured to fluidly couple the inlet and the outlet when a pressure force from the first portion of the eye exceeds a threshold value, wherein fluid coupling of the inlet and the outlet enables drainage of the fluids from the first portion of the eye to the second portion of the eye to maintain the IOP.

### INTRODUCTION

Postoperative intraocular pressure (IOP) elevation is typically treated with eye drops and/or oral medication. Eye drops add to the post-operative burden associated with patient eye care, while oral medications may have side effects. In addition, both treatments are sometimes ineffective. Glaucoma filtering surgery may be performed as a last resort.

### SUMMARY

This summary is provided to introduce a selection of concepts that are further described below in the Detailed Description. This summary is not intended to identify key or essential features of the claimed subject matter, nor is it to be used as an aid in limiting the scope of the claimed subject matter.

A device for maintaining an intraocular pressure (IOP) in an eye. The device includes: an inlet configured to receive fluids from a first portion of the eye; an outlet configured to drain the fluids into a second portion of the eye; and a pressure regulator disposed between the inlet and the outlet, the pressure regulator comprising a pressure resistance apparatus configured to fluidly couple the inlet and the outlet when a pressure force from the first portion of the eye exceeds a threshold value, wherein fluid coupling of the inlet and the outlet enables drainage of the fluids from the first portion of the eye to the second portion of the eye to maintain the IOP.

### BRIEF DESCRIPTION OF THE DRAWINGS

A more complete understanding of the subject matter of the present disclosure may be obtained by reference to the following Detailed Description when taken in conjunction with the accompanying Drawings wherein:

FIGS. 1A-1C illustrate example geometries and placements of intraocular pressure (IOP) maintenance devices inserted within an eye according to aspects of the disclosure.

FIG. 2 illustrates a schematic representation of an IOP-maintaining mechanism of an IOP maintenance device of any of FIGS. 1A-1C inserted within the eye according to aspects of the disclosure.

FIG. 3A illustrates a cross-sectional side view of an example IOP maintenance device according to aspects of the disclosure.

FIG. 3B illustrates a perspective top view of the IOP maintenance device of FIG. 3A according to aspects of the disclosure.

FIG. 3C illustrates a perspective bottom view of the IOP maintenance device of FIG. 3A according to aspects of the disclosure.

FIG. 4A illustrates a cross-sectional side view of an example IOP maintenance device according to aspects of the disclosure.

FIG. 4B illustrates a perspective top view of the IOP maintenance device of FIG. 4A according to aspects of the disclosure.

FIG. 4C illustrates a perspective bottom view of the IOP maintenance device of FIG. 4A according to aspects of the disclosure.

FIG. 5 illustrates a cross-sectional side view of an example IOP maintenance device according to aspects of the disclosure.

FIG. 6A illustrates an enlarged cross-sectional side view of an embodiment of the first and second tapered portions of FIGS. 3A-C, 4A-C, and 5.

FIG. 6B illustrates an enlarged cross-sectional side view of an embodiment of the first and second tapered portions of FIGS. 3A-C, 4A-C, and 5.

### DETAILED DESCRIPTION

It is to be understood that the following disclosure provides many different embodiments, or examples, for implementing different features of various embodiments. Specific examples of components and arrangements are described below to simplify the disclosure. These are, of course, merely examples and are not intended to be limiting. The section headings used herein are for organizational purposes and are not to be construed as limiting the subject matter described.

Intraocular pressure (IOP) is the pressure, or force, inside of the eyes. Postoperative IOP fluctuations are differences between maximal and minimal IOP values measured during a day and may be an undesired complication after ophthalmic surgeries that can lead to blindness by progressive and irreversible damage to a nerve in the back of the eye called the optic nerve.

Glaucoma is a group of eye diseases that can cause vision loss and blindness by damaging the optic nerve. Normal IOP is in a range of approximately 10 to 21 millimeters of mercury (mmHg), but it can drop to as low as 0 mmHg in hypotony and can exceed 70 mmHg in some glaucomas. Glaucoma is currently the second leading cause of worldwide blindness, after cataracts, with an incidence rate of approximately 2.5 million per year (approximately 4 million North Americans are affected by glaucoma). The worldwide glaucoma market is dominated by topical and oral drug therapies, which represents approximately 95% of the total market. The remaining approximately 5% is accounted for by minimally invasive surgical implants and/or major ophthalmic surgeries. A primary risk factor associated with surgical intervention of glaucoma is elevated IOP as caused by, for example, postoperative IOP fluctuation.

Currently, postoperative IOP elevation is treated initially with topical drops—which add to the post-operative burden associated with patient eye care. The most widely used topical drugs include, but are not limited to, prostaglandin analogs (which increases aqueous humor drainage from the eye), alpha agonist, beta blockers, and carbonic anhydrase inhibitors (which decrease the aqueous humor production). Topical medications can decrease IOP, on average, by approximately 15%, but have an array of side effects and contraindications.

When topical medications are not sufficient, oral medications such as acetazolamide and methazolamide may be utilized, which can also have undesired side effects similar to topical treatments. And, if topical and/or oral medications fail, glaucoma filtering surgery may be performed as a last resort measure. Yet, current glaucoma filtering surgeries have their own postoperative complications including, for example, hypotony, serous and hemorrhagic choroidal detachments, and the like.

In addition to the complications noted above, none of the current treatments for postoperative IOP elevation, medical and/or surgical, have the capability of maintaining the IOP at a given predetermined value (e.g., managing the fluctuations of IOP) for extended periods of time, e.g., for the duration of a postoperative recovery period.

The present disclosure describes examples of IOP maintenance devices that can prevent and/or relieve elevated postoperative IOP, thereby enabling maintenance of postoperative IOP at desired levels or values (e.g., a threshold value of 21 mmHg is commonly used for current treatments) for extended periods of time, such as for the duration of a postoperative recovery period of a glaucoma surgery patient. In certain embodiments, the IOP maintenance devices presented herein also have many other advantages over traditional treatment modalities. For example, in certain embodiments, the IOP maintenance devices disclosed herein are designed for temporary use and are easily implantable intraoperatively and easily removable postoperatively (e.g., in-clinic based on the vitreoretinal surgeon's judgement). In further embodiments, the IOP maintenance devices disclosed herein eliminate the need for additional IOP lowering intervention, which can improve patient postoperative clinical care, and thus, quality of life. And, in certain embodiments, the IOP maintenance devices disclosed herein are compatible with one or more types of vitreous humor and/or aqueous humor substitutes (e.g., air, gas, silicone oil, etc.) that may be used during surgical procedures to maintain IOP.

The IOP maintenance devices disclosed herein can be implanted by a surgeon intraoperatively, for example, either in the anterior chamber or the pars plana, and can be kept implanted during the postoperative course. Once the risk of IOP elevation is determined to be minimal (or eliminated) by the surgeon, the IOP maintenance devices can be safely removed in-clinic with a slit-lamp, thus omitting the need for further intraoperative procedures. Moreover, the IOP maintenance devices may be designed to be small enough to allow for scleral self-sealing once removed.

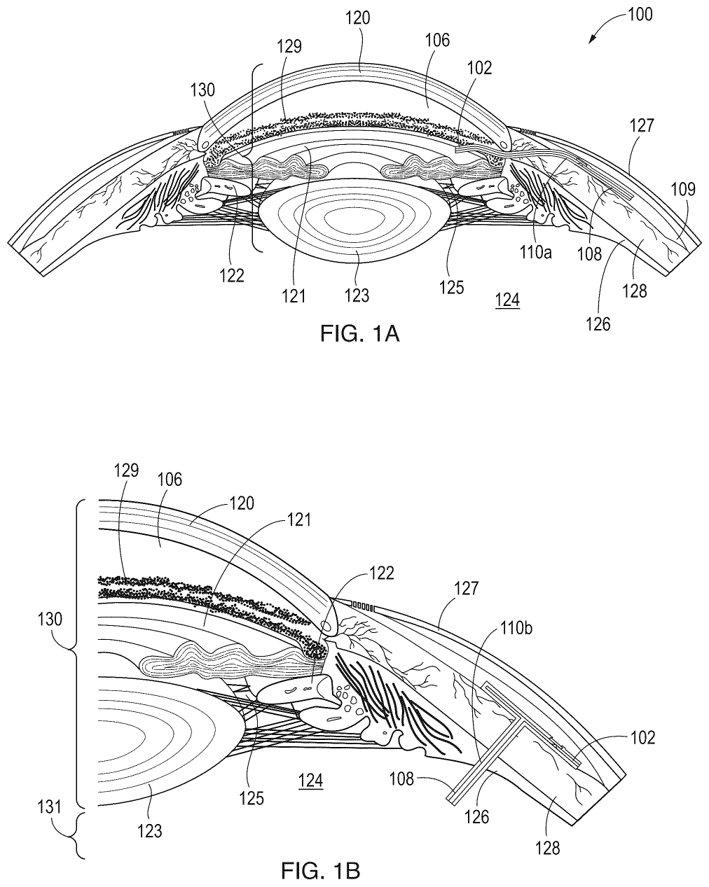

FIGS. 1A-1C illustrate example geometries and placement of IOP maintenance devices 110a, 110b, and 110c (collectively referred to as an IOP maintenance device 110 unless specifically referenced) inserted within an eye 100 according to aspects of the disclosure. FIGS. 1A-1C are described together for clarity.

As shown in FIGS. 1A-1C, the IOP maintenance device 110 may be inserted into the eye 100 such that an inlet 102 of the IOP maintenance device 110 is exposed to IOP inside the eye 100. IOP is naturally maintained, for example, by the production and drainage of aqueous humor by a ciliary body 122, as well as the drainage of aqueous humor via a trabecular meshwork 129 and uveoscleral outflow (i.e., the drainage of aqueous humor from an anterior chamber 106 into portions of the eye 100 other than through the trabecular meshwork 129). It should be understood that while IOP is described herein as being elevated as a result of disproportionate production and drainage of aqueous humor, other fluids within the eye may also increase IOP.

In certain embodiments, the inlet 102 may be exposed to the IOP via a portion of an anterior segment 130. The anterior segment 130 includes, for example, areas in front of a vitreous cavity 124 (e.g., cornea 120, iris 121, ciliary body 122 and/or lens 123), a pars plana 126, as well as spaces of the anterior chamber 106 and/or posterior chamber 125 filled with aqueous humor and/or other fluid. The anterior chamber 106 is between the posterior surface of the cornea 120 (i.e., the corneal endothelium) and the iris 121, and the posterior chamber 125 is between the iris 121 and the front face of the vitreous cavity 124. Thus, in certain embodiments, the IOP maintenance device 110 may be implanted intraoperatively such that inlet 102 is disposed in or exposed to the anterior chamber 106, posterior chamber 125, or other areas of anterior segment 130 experiencing elevated IOP.

In certain embodiments, as shown in FIGS. 1B-1C, the IOP maintenance device may be exposed to the IOP via a portion of a posterior segment 131. The posterior segment 131 includes, for example, the vitreous cavity 124. Thus, in certain embodiments, the IOP maintenance device 110 may be implanted intraoperatively such that the inlet 102 is disposed in or exposed to the vitreous cavity 124 or other areas of the posterior segment 131 to drain fluids therefrom.

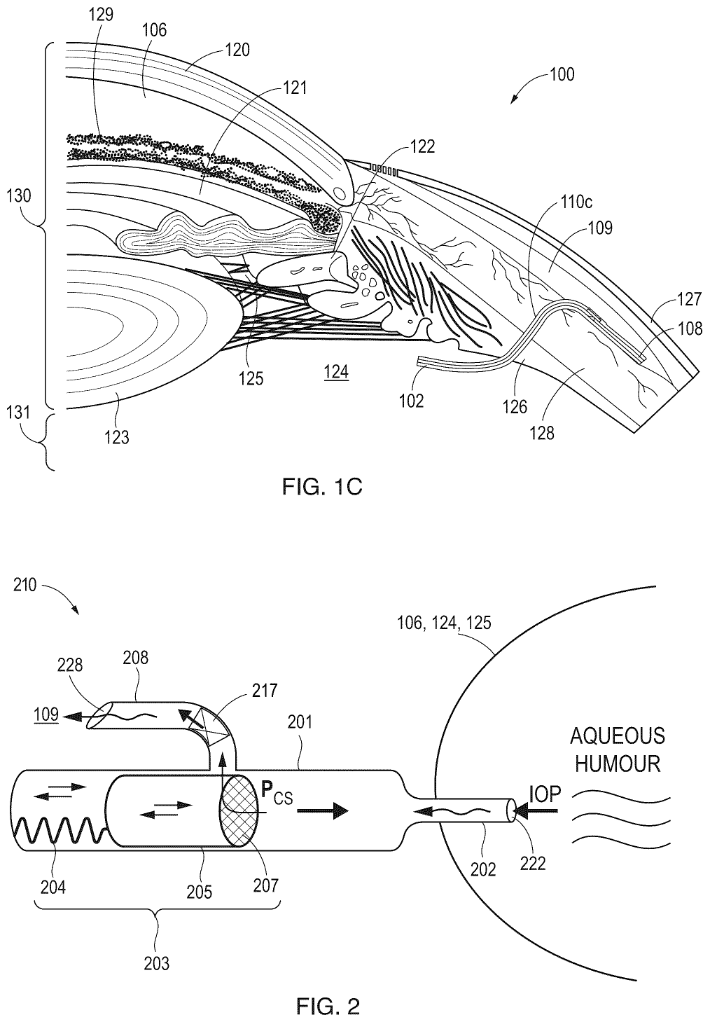

The IOP maintenance device 110 includes an outlet 108 to facilitate the maintenance, or fixation, of the IOP at a desired value within the anterior segment 130 or posterior segment 131 by removal of aqueous humor and/or other fluids to a subconjunctival space 109. The subconjunctival space may include, for example, the hydrophilic, fluid-filled space between a conjunctiva 127 and a sclera 128 of the eye 100. Operation of the outlet 108 is described in further detail below with respect FIG. 2.

The IOP maintenance device 110 may include various geometries and insertion placements as illustrated in FIGS. 1A-1C. For example, and not by way of limitation, the IOP maintenance device 110a, as shown in FIG. 1A, may have a geometry that includes a semi-linear geometry facilitating the fluidic coupling of the outlet 108 in the subconjunctival space 109 with the inlet portion 102 in the anterior chamber 106, the posterior chamber 125, or the vitreous cavity 124. In FIG. 1A, IOP maintenance device 110a is implanted through the sclera 128, the ciliary body 122, and the trabecular meshwork 129 to fluidically couple the anterior chamber 106 and the subconjunctival space 109.

In another example, the IOP maintenance device 110b, as shown in FIG. 1B, may have a T-shaped geometry such that the outlet 108 at least partly forms the upper portion of the T-shape along the sclera 128, and the lower portion of the T-shape is formed at least in part by the inlet 102, which is exposed to the anterior segment 130 or vitreous cavity 124. In FIG. 1B, IOP maintenance device 110b is implanted through the sclera 128 and the pars plana 126 to fluidically couple the vitreous cavity 124 and the subconjunctival space 109.

In certain embodiments, the IOP maintenance device 110c, as shown in FIG. 1C, may have a curved, S-shaped geometry with the outlet 108 forming at least a part of the upper portion of the S-shape and the inlet 102 at least partly forming the lower portion of the S-shape entering the anterior segment 130 or vitreous cavity 124. In FIG. 1C, IOP maintenance device 110c is implanted through the sclera 128 and the pars plana 126 to fluidically couple the vitreous cavity 124 and the subconjunctival space 109.

In various embodiments, the geometry and/or placement of the IOP maintenance device 110 may include a combination of one or more geometries and/or placements of the IOP maintenance devices 110a, 110b, and/or 110c, as illustrated in FIGS. 1A-1C.

While various geometries and placements are illustrated with respect to FIGS. 1A-1C, other geometries and/or placement of the IOP maintenance device 110 are readily envisioned. Such geometries may include, without limitation, any shape, form, and/or size that allow the outlet 108 to drain aqueous humor and/or other fluids into the subconjunctival space 109 via the inlet 102, which is exposed to at least a portion of the anterior segment 130 (e.g., the anterior chamber 106 or the posterior chamber 125) or vitreous cavity 124. For example, an L-shaped or C-curved geometry is readily envisioned for the IOP maintenance device 110, according to certain embodiments.

In certain embodiments, inlet 102, outlet 108, and/or the IOP maintenance device 110 may be designed to be small enough to allow for scleral self-sealing once removed. In certain embodiments, inlet 102, outlet 108, and/or the IOP maintenance device 110 as a whole may have an outer diameter size in the range of approximately 0.1 to 1 millimeter (mm). In certain embodiments, inlet 102, outlet 108, and/or the IOP maintenance device 110 may have an outer diameter size in the range of approximately 0.159 to 0.566 mm. In other embodiments, inlet 102, outlet 108, and/or the IOP maintenance device 110 as a whole may have an outer diameter size in the range of approximately 0.159 to 0.312 mm. In some embodiments, inlet 102, outlet 108, and/or the IOP maintenance device 110 as a whole may have an outer diameter size less than 0.159 mm. In some embodiments, inlet 102, outlet 108, and/or the IOP maintenance device 110 as a whole may have an outer diameter size greater than 0.159 mm.

Additionally, the IOP maintenance device 110 may be composed of one or more of a variety of materials to facilitate in proper alignment and/or placement geometry. For example, the materials of the IOP maintenance device 110 may include, without limitation, plastics, metals (e.g., titanium, titanium alloys, steel, and/or steel alloys), polymers (e.g., polyvinylchloride, polyethylene, polypropylene, polytetrafluoroethylene, polymethylmethacrylate, and/or trimethylcarbonate), ceramic biomaterials (e.g., alumina, bioglass, cobalt-chromium alloys, zirconia, and/or hydroxyapatite), gels (e.g., gelatin), fabrics, biomaterials, biocompatible materials, silicone, soft semi-rigid materials, flexible materials, and combinations of the same and like.

FIG. 2 schematically illustrates an example configuration of an IOP maintenance device 210 when inserted within the eye 100 according to aspects of the disclosure. IOP maintenance device 210 may be representative of the IOP maintenance device 110a, 110b, and 110c of FIGS. 1A-1C.

The IOP maintenance device 210 includes a pressure regulator 201 fluidically coupled to an inlet 202 and an outlet 208. In the example of FIG. 2, the inlet 202 has a tube-like structure, which may be formed of rigid or flexible materials, though other geometries are also contemplated. A proximal end of the inlet 202 is fluidly coupled to the pressure regulator 201, and a distal end of the inlet 202 is configured to be disposed in, e.g., the anterior chamber 106, posterior chamber 125, or vitreous cavity 124 to receive flow of aqueous humor and/or other fluids through a distal opening 222.

Similar to the inlet 202, the outlet 208 in the example of FIG. 2 has a tube-like structure, which may be formed of rigid or flexible materials, though other geometries are also contemplated. A distal end of the outlet 208 is in fluid communication with the pressure regulator 201, while a proximal end of the outlet 208 is configured to be disposed in the subconjunctival space 109, or another portion of the eye 100, for drainage of fluids through a proximal opening 228.

In certain embodiments, the IOP maintenance device 210 further includes a valve 217, (e.g., a one-way safety valve or other single-direction flow valve) disposed within the outlet 208. In certain embodiments, the valve 217 is disposed at, or near, a junction between the pressure regulator 201 and the distal end of the outlet 208. The valve 217 restricts passage of fluid out of pressure regulator 201 and into the outlet 208. In certain embodiments, the valve 217 blocks the reflux of fluid from the outlet 208 into the pressure regulator 201 in order facilitate or maintain a constant IOP.

A pressure resistance apparatus 203 is disposed within the pressure regulator 201 to regulate the flow of fluids between the inlet 202 and the outlet 208. In the example of FIG. 2, the pressure resistance apparatus 203 includes an elastic device 204 extending from an inside surface of the pressure regulator 201 and coupled to a slidable plunger 205. In certain embodiments, the elastic device 204 and the plunger 205 may form a single unit that moves back and forth within the pressure regulator 201 to regulate fluid flow. In other embodiments, the elastic device 204 and the plunger 205 may form multiple units that move back and forth within the pressure regulator 201 to regulate fluid flow. The pressure regulator 201 may generally have a tube-like geometry, or any other suitable geometry, to support the translation of the plunger 206 and/or elastic device 204.

During use, the elastic device 204 generates a distal (e.g., towards the inlet 202) biasing force against the plunger 205. In certain embodiments, the biasing force facilitates maintenance of the IOP in, e.g., the anterior chamber 106, the posterior chamber 125, or the vitreous cavity 124 of the eye 100. The biasing force may be, or can include, a pressure force (P_{CS}) within the pressure regulator 201 acting in opposition to the pressure forces created by IOP (such pressure forces herein referred to as “the IOP” for clarity). Accordingly, in certain embodiments, the IOP maintenance device 110 operates based on a pressure differential between the IOP and the biasing force of the elastic device 204.

For example, when the IOP is greater than P_{CS} (e.g., IOP>P_{CS}), a net positive proximal force (from, e.g., the anterior chamber 106, the posterior chamber 125, or the vitreous cavity 124) is formed, which acts on plunger 205 and moves plunger 205 proximally (e.g., away from the inlet 202). The proximal movement of the plunger 205 at least partially exposes the outlet 208 to fluidly couple the inlet 202 and the outlet 208. The exposure or opening of the outlet 208 to the inlet 202, allows aqueous humor and/or other fluids to flow from, e.g., the anterior chamber 106, the posterior chamber 125, or the vitreous cavity 124 through the inlet 202, through the outlet 208, and out of the proximal opening 228 and into, e.g., the subconjunctival space 109. This configuration is herein referred to as an “open” position of the pressure resistance apparatus 203 and is maintained for as long as the IOP is greater than P_{CS}.

When the IOP is equal to or less than P_{CS} (e.g., IOP≤P_{CS}), a net positive distal force from the elastic device 204 acts on plunger 205 and moves it distally (e.g., toward the inlet 202). The distal movement of the plunger 205 covers or seals the outlet 208 from the inlet 202, thereby stopping or preventing the flow of fluids between the inlet 202 and the outlet 208. This configuration is herein referred to as a “closed” position of the pressure resistance apparatus 203 and is maintained for as long as the IOP is equal to or less than P_{CS}. Thus, when enough aqueous humor and/or other fluids have been drained during an episode of elevated IOP, the IOP equalizes with P_{CS} and the plunger 205 moves back to the closed position to prevent further fluid drainage from, e.g., the anterior chamber 106, the posterior chamber 125, or the vitreous cavity 124 to, e.g., the subconjunctival space 109.

In the example of FIG. 2, the elastic device 204 is disposed in an expanded and/or resting state in the closed position of pressure resistance apparatus 203. In the open position of pressure resistance apparatus 203, the elastic device 204 is disposed in a compressed state. However, it is further contemplated that other types of states, movements, manipulations, or distortions of the elastic device 204 may be utilized to open and/or close the outlet 208 to the inlet 202.

In certain embodiments, P_{CS} is based on a resistance and/or an elasticity of the elastic device 204, which may be changed by altering the elastic device 204. Accordingly, the elastic device 204 may be designed or selected such that the biasing force provided by the elastic device 204, e.g., an elasticity of the elastic device 204, may correspond to target values, or ranges of values, for IOP. That way, the IOP maintenance device 110 may be configured and/or selected to maintain a threshold value of the IOP within the eye 100 and lower the IOP to the threshold value during an episode of elevated IOP. For example, in patients with glaucoma or fragile optic nerves, the IOP maintenance device 110 may be selected such as to mitigate postoperative elevated IOP by, for example, maintaining the IOP at or below a fixed threshold value consistently and/or constantly. For example, the elastic device 204 may be chosen to maintain the IOP at a pressure level in a range of approximately 11 to 21 mmHg.

In certain embodiments, the elastic device 204 may include, without limitation, a spring such as a compression spring, an elastomer, an elastic material, rubber, isoprene, neoprene, synthetic polymers, and/or a combination of one or more of the same and like. In certain embodiments, the elastic device 204 may be any material that exhibits elasticity (i.e., the ability to resume the normal shape after being stretched and/or compressed). In embodiments where the elastic device 204 comprises a spring, the pressure force P_{CS} acting in opposition to the IOP is based, at least in part, on at least one of a number of turns within a coiled portion of the spring or a thickness of the spring. In certain embodiments, the elasticity and/or pressure force P_{CS} provided by the elastic device 204 may be adjusted via a mechanism built into the IOP maintenance device 110. For example, in some embodiments, a setscrew or other control means may be used to adjust the spring (e.g., tightness or number of turns of the coiled portion of the spring) or other elastic device.

Generally, the plunger 205 includes a non-permeable material 207 on at least a distal end of the plunger 205 opposite the elastic device 204. The non-permeable material 207 prevents fluid from soaking and infiltrating the plunger 205. In certain embodiments, the entirety of the plunger 205 may be formed of the non-permeable material 207.

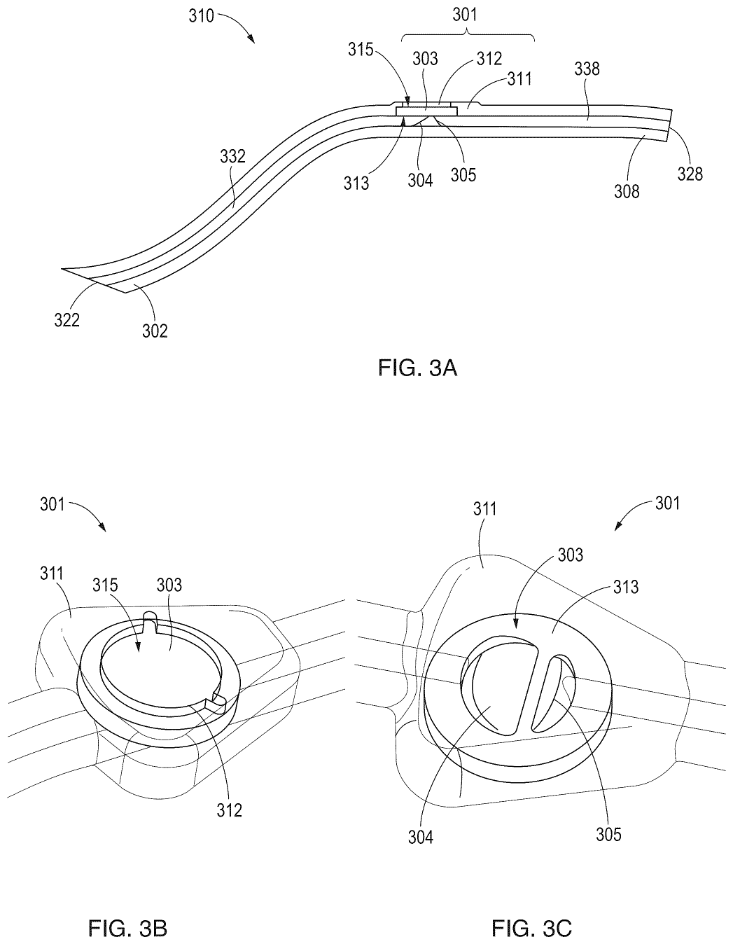

FIGS. 3A-3C illustrate an example configuration of an IOP maintenance device 310 according to aspects of the disclosure. IOP maintenance device 310 may be representative of the IOP maintenance devices 110a and 110b of FIGS. 1A-1B. FIG. 3A illustrates a cross-sectional side view of the IOP maintenance device 310 according to aspects of the disclosure. FIG. 3B illustrates a perspective top view of the IOP maintenance device 310 according to aspects of the disclosure. FIG. 3C illustrates a perspective bottom view of the IOP maintenance device 310 according to aspects of the disclosure. FIGS. 3A-3C will be described together for clarity.

As shown, the IOP maintenance device 310 includes a pressure regulator 301 fluidically coupled to an inlet 302 and an outlet 308. In the example of FIGS. 3A-3C, the inlet 302 has a tube-like structure, which may be formed of rigid or flexible materials, though other geometries are also contemplated. A distal end of the inlet 302 is configured to be disposed in, e.g., the anterior chamber 106, posterior chamber 125, or vitreous cavity 124 of the eye 100 to receive flow of aqueous humor and/or other fluids through a distal opening 322, and a proximal end of the inlet 302 is fluidly coupled to the pressure regulator 301. A passage 332 extends between and fluidly couples the distal opening 322 and the pressure regulator 301. Generally, the inlet 302 may be composed of polymers, plastics (e.g., thermoplastics), rubbers, metals (e.g., alloys), composites (e.g., plastic composites), elastic materials, or the like.

Similar to the inlet 302, the outlet 308 in the example of FIGS. 3A-3C has a tube-like structure, which may be formed of rigid or flexible materials, though other geometries are also contemplated. A proximal end of the outlet 308 is configured to be disposed in the subconjunctival space 109, or another portion of the eye 100, for drainage of fluids through a proximal opening 328 of the outlet 308, and a distal end of the outlet 308 is in fluid communication with the pressure regulator 301. A passage 338 extends between and fluidly couples the proximal opening 328 and the pressure regulator 301. Generally, the outlet 308 may be composed of polymers, plastics (e.g., thermoplastics), rubbers, metals (e.g., alloys), composites (e.g., plastic composites), elastic materials, or the like.

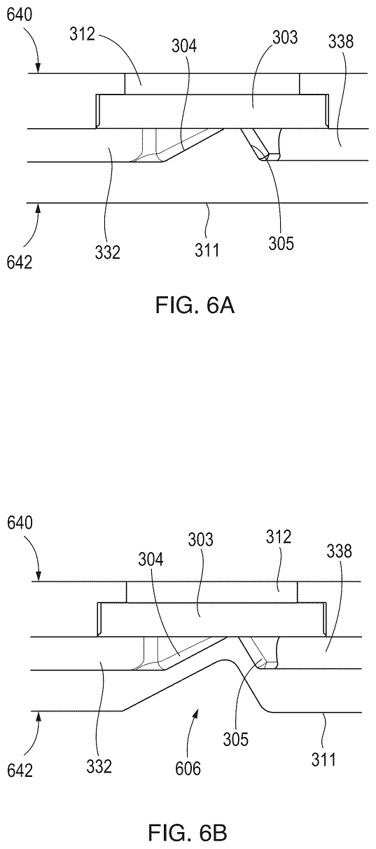

A pressure resistance apparatus is disposed within the pressure regulator 301 to regulate the flow of fluids between the inlet 302 and the outlet 308. In the example of FIGS. 3A-3C, the pressure resistance apparatus includes a deformable diaphragm 303 that is disposed between the inlet 302 and the outlet 308 and is supported by a housing 311 of the pressure regulator 301. The passage 332 of the inlet 302 leads into a first side 313 of the diaphragm 303 at a first tapered portion 304 of the passage 332 in the housing 311 (discussed in further detail with respect to FIG. 6), and the passage 338 of the outlet 308 leads out from the first side 313 of the diaphragm 303 at a second tapered portion 305 of the passage 338 in the housing 311 (discussed in further detail with respect to FIG. 6). The diaphragm 303 is configured to deform, or bend, upon application of a pressure force greater than a pressure or flexure threshold (F_{T}) of the diaphragm 303. To facilitate deformation of the diaphragm 303, a second side 315 of the diaphragm 303 is disposed adjacent to an opening 312 in the pressure regulator 301 that can accommodate the diaphragm 303 when deformed. In some embodiments, the diaphragm 303 may be composed of one or more of a variety of materials, such as polymers, plastics (e.g., thermoplastics), rubbers, metals (e.g., alloys), composites (e.g., plastic composites), elastic materials, or the like.

The housing 311 may have a configuration operable to surround at least part of the diaphragm 303, thereby keeping the diaphragm in place between the inlet 302 and the outlet 308. In certain embodiments, the housing 311 may have any suitable shape for implantation into a desired cavity and/or tissue within the eye (e.g., triangular as shown in FIGS. 3B-C). Additionally and/or alternatively, the housing 311 may have a configuration operable to assist in anchoring the IOP maintenance device 310 in the eye. In certain embodiments, the housing 311 may be composed of one or more of a variety of materials, such as polymers, plastics (e.g., thermoplastics), rubbers, metals (e.g., alloys), composites (e.g., plastic composites), elastic materials, and the like.

During use, fluids flow from the eye 100 and into the distal opening 322, through the passage 332 of the inlet 302, and to the diaphragm 303. When the IOP is at or below the flexure threshold of the diaphragm 303 (e.g., IOP≤F_{T}), the diaphragm 303 maintains a resting or non-deformed state, wherein the first side 313 of the diaphragm 303 seals or blocks fluid flow between the inlet 302 and the outlet 308. During episodes of elevated IOP, pressure forces created by the IOP (such pressure forces herein referred to as “the IOP” for clarity) can build against the first side 313 of the diaphragm 303 at the first tapered portion 304. When the IOP built up at the first tapered portion 304 exceeds F_{T} of the diaphragm 303, the diaphragm 303 deforms, or bends, to allow fluid to bypass the diaphragm 303 and flow into the passage 338 of the outlet 308 through the second tapered portion 305. The deformation of the diaphragm 303 thus allows aqueous humor and/or other fluids to flow from, e.g., the anterior chamber 106, the posterior chamber 125, or the vitreous cavity 124 through the inlet 302, out of the outlet 308, and into, e.g., the subconjunctival space 109 or other portions of the eye 100. The diaphragm 303 may, therefore, provide similar functionality to the plunger 205 and elastic device 204 of FIG. 2. A deformed state of the diaphragm 303 may thus correspond to an “open” configuration of the IOP maintenance device 310, while a non-deformed state of the diaphragm 303 may correspond to a “closed” configuration of the IOP maintenance device 310.

In certain embodiments, F_{T} is based on a flexural strength of the diaphragm 303, which may be changed by altering the diaphragm 303. Accordingly, the diaphragm 303 may be designed or selected such that the F_{T} of the diaphragm 303 may correspond to target values, or ranges of values, for IOP. That way, the IOP maintenance device 310 may be configured and/or selected to maintain a threshold value of the IOP within the eye 100 and lower the IOP to the threshold value during an episode of elevated IOP. For example, in patients with glaucoma or fragile optic nerves, the IOP maintenance device 310 may be selected such as to mitigate postoperative elevated IOP by, for example, maintaining the IOP at or below a fixed threshold value consistently and/or constantly. For example, the diaphragm 303 may be chosen to maintain the IOP at a pressure level in a range of approximately 11 to 21 mmHg.

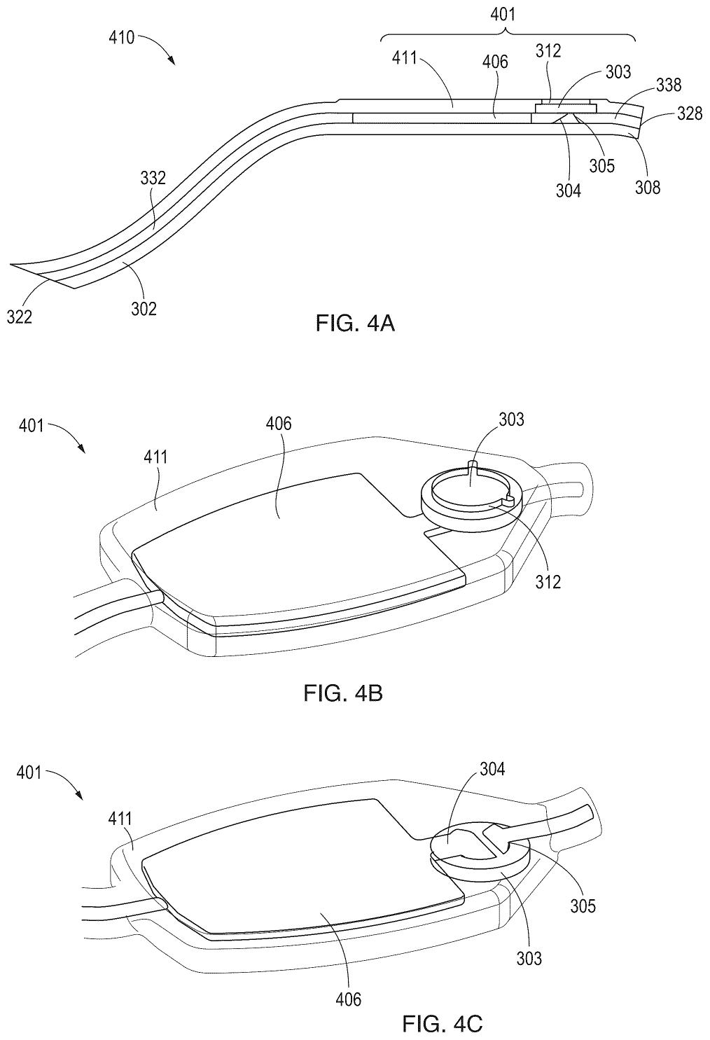

FIGS. 4A-4C illustrate an example configuration of an IOP maintenance device 410 according to aspects of the disclosure. IOP maintenance device 410 may be representative of the IOP maintenance devices 110a and 110b of FIGS. 1A-1B. FIG. 4A illustrates a cross-sectional side view of IOP maintenance device 410 according to aspects of the disclosure. FIG. 4B illustrates a perspective top view of the IOP maintenance device 410 according to aspects of the disclosure. FIG. 4C illustrates a perspective bottom view of the IOP maintenance device 410 according to aspects of the disclosure. FIGS. 4A-4C will be described together for clarity.

As shown, the IOP maintenance device 410 is substantially similar to IOP maintenance device 310 and includes many of the same or similar features having the same or similar structure and functionality as described above with reference to the IOP maintenance device 310. Accordingly, such features are not described herein, and are illustrated with the same reference numerals.

Unlike the IOP maintenance device 310, the IOP maintenance device 410 includes a pressure regulator 401 having a housing 411 with a reservoir 406 disposed therein. The reservoir 406 is disposed within the housing 411 such that it divides, or splits, the passage 338 from the first tapered portion 304. Thus, instead of the passage 338, the reservoir 406 leads into the diaphragm 303 via the first tapered portion 304 (discussed in further detail with respect to FIG. 6), while the outlet 308 leads out of the diaphragm 303 at the second tapered portion 305 (discussed in further detail with respect to FIG. 6).

During use, as fluids flow from the eye 100 and into the IOP maintenance device 410, the reservoir 406 fills with such fluids. Only when the reservoir 406 reaches capacity does pressure start to build up on the diaphragm 303 as a result of the incoming fluids, as described above with reference to IOP maintenance device 310. In this manner, the reservoir 406 may provide a time delay equal to the time to reach capacity, before the diaphragm 303 deforms to relieve elevated pressure. Such functionality may be beneficial for short fluctuations of IOP, wherein IOP may be elevated for only short periods of time before equalizing or decreasing and potentially leading to hypotony.

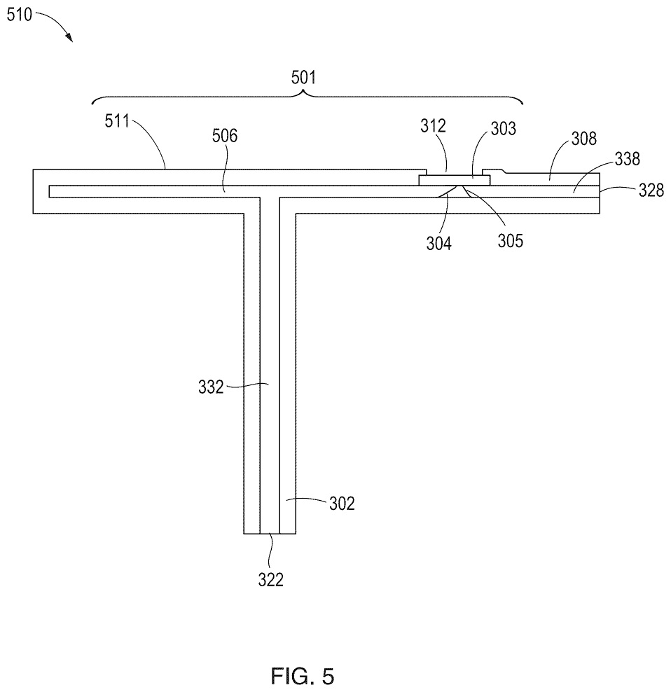

FIG. 5 illustrates an example configuration of an IOP maintenance device 510 according to aspects of the disclosure. More particularly, FIG. 5 illustrates a cross-sectional side view of IOP maintenance device 410 according to aspects of the disclosure. IOP maintenance device 510 may be representative of the IOP maintenance device 110c of FIG. 1C.

The IOP maintenance device 510 is similar to IOP maintenance devices 310 and 410 and includes many of the same or similar features having the same or similar structure and functionality as described above with reference to the IOP maintenance devices 310 and 410. Accordingly, such features are not described herein, and are illustrated with the same reference numerals.

The IOP maintenance device 510 includes a pressure regulator 501 having a housing 511 with a reservoir 506 disposed therein, similar to the IOP maintenance device 410. However, unlike the IOP maintenance device 410, the inlet 302 couples to and extends from the housing 511 (and thus, the reservoir 506) at a “bottom” portion of the housing 511, rather than a lateral portion of the housing 511. In certain embodiments, the inlet 302 may extend from the housing 511 at an orthogonal angle relative to a major axis or length of the housing 511 and the outlet 308. Accordingly, this arrangement of the inlet 302 and the housing 511, as shown in FIG. 5, can form a T-shaped geometry similar to that of the example in FIG. 1C, such that the housing 511 (including the reservoir 506) and the outlet 308 at least partly form the upper portion of the T-shape, and the lower portion of the T-shape is formed at least in part by the inlet 302. A T-shaped geometry may be particularly beneficial for draining fluids from, and maintaining the IOP of, the vitreous cavity 124.

In another example, the IOP maintenance device 110b, as shown in FIG. 1B, may have a T-shaped geometry such that the outlet 108 at least partly forms the upper portion of the T-shape along the sclera 128, and the lower portion of the T-shape is formed at least in part by the inlet 102, which is exposed to the anterior segment 130 or vitreous cavity 124. In FIG. 1B, IOP maintenance device 110b is implanted through the sclera 128 and the pars plana 126 to fluidically couple the vitreous cavity 124 and the subconjunctival space 109.

FIG. 6A illustrates an enlarged cross-sectional side view of an embodiment of the first and second tapered portions 304, 305 of FIGS. 3A-C, 4A-C, and 5. FIG. 6B illustrates an enlarged cross-sectional side view of another embodiment of the first and second tapered portions 304, 305 of FIGS. 3A-C, 4A-C, and 5. FIGS. 6A-6B will be described together for clarity.

As illustrated in FIGS. 6A-6B, passage 332 of the inlet 302 leads into the diaphragm 303 at the first tapered portion 304, and the passage 338 of the outlet 308 leads out of the diaphragm 303 at the second tapered portion 305. The diaphragm 303 seals off fluid communication between the first tapered portion 304 and the second tapered portion 305. The diaphragm 303 is disposed within the housing 311, as described above, which has the opening 312 formed in a top side 640 of the housing 311 to enable the diaphragm 303 to deform when pressure is applied to the diaphragm 303. In FIG. 6A, the housing 311 has a substantially planar or linear bottom side 642 disposed opposite of the top side 640.

With particular attention to FIG. 6B, a cavity 606 may be formed in the bottom side 642 of the housing 311. In certain embodiments, the cavity 606 may mirror, partially or fully, the angles or slopes of first tapered portion 304 and/or the second tapered portion 305 within the housing 311. In certain embodiments, the cavity 606 may assist in facilitating the placing of the IOP maintenance device within the eye 100. In certain embodiments, the cavity 606 may decrease overall weight of the IOP maintenance device. In certain embodiments, the cavity 606 may reduce space of the IOP maintenance device in the eye. Additionally and/or alternatively, the cavity 606 may be configured to provide anchoring and/or support of the IOP maintenance device in the eye. Accordingly, the cavity 606 may have any suitable geometry and/or taper leading to the diaphragm 303 to facilitate in securing and/or placing the IOP maintenance device within the eye 100.

Although various embodiments of the present disclosure have been illustrated in the accompanying Drawings and described in the foregoing Detailed Description, it will be understood that the present disclosure is not limited to the embodiments disclosed herein, but is capable of numerous rearrangements, modifications, and substitutions without departing from the spirit of the disclosure as set forth herein.

The term “substantially” is defined as largely but not necessarily wholly what is specified, as understood by a person of ordinary skill in the art. In any disclosed embodiment, the terms “substantially”, “approximately”, “generally”, and “about” may be substituted with “within [a percentage] of” what is specified, where the percentage includes 0.1, 1, 5, and 10 percent.

The foregoing outlines features of several embodiments so that those of ordinary skill in the art may better understand the aspects of the disclosure. Those of ordinary skill in the art should appreciate that they may readily use the disclosure as a basis for designing or modifying other processes and structures for carrying out the same purposes and/or achieving the same advantages of the embodiments introduced herein. Those of ordinary skill in the art should also realize that such equivalent constructions do not depart from the spirit and scope of the disclosure, and that they may make various changes, substitutions, and alterations herein without departing from the spirit and scope of the disclosure. The scope of the invention should be determined only by the language of the claims that follow. The term “comprising” within the claims is intended to mean “including at least” such that the recited listing of elements in a claim are an open group. The terms “a”, “an”, and other singular terms are intended to include the plural forms thereof unless specifically excluded.

Conditional language used herein, such as, among others, “can”, “might”, “may”, “e.g.”, and the like, unless specifically stated otherwise, or otherwise understood within the context as used, is generally intended to convey that certain embodiments include, while other embodiments do not include, certain features, elements, and/or states. Thus, such conditional language is not generally intended to imply that features, elements, and/or states are in any way required for one or more embodiments.

While the above detailed description has shown, described, and pointed out novel features as applied to various embodiments, it will be understood that various omissions, substitutions, and changes in the form and details of the embodiments illustrated can be made without departing from the spirit of the disclosure. As will be recognized, the various embodiments described herein can be embodied within a form that does not provide all of the features and benefits set forth herein, as some features can be used or practiced separately from others. The scope of protection is defined by the appended claims rather than by the foregoing description. All changes which come within the meaning and range of equivalency of the claims are to be embraced within their scope.

What is claimed is:

1. A device for maintaining an intraocular pressure (IOP) in an eye, the device comprising:
an inlet configured to receive fluids from a first portion of the eye;
an outlet configured to drain the fluids into a second portion of the eye; and
a pressure regulator disposed between the inlet and the outlet, the pressure regulator comprising a pressure resistance apparatus configured to fluidly couple the inlet and the outlet when a pressure force from the first portion of the eye exceeds a threshold value, wherein fluid coupling of the inlet and the outlet enables drainage of the fluids from the first portion of the eye to the second portion of the eye to maintain the IOP.

2. The device of claim 1, wherein the inlet comprises a tube configured to be at least partially disposed in an anterior chamber, a posterior chamber, or a vitreous cavity of the eye.

3. The device of claim 1, wherein the outlet comprises a tube configured to be disposed in a subconjunctival space of the eye.

4. The device of claim 1, wherein the inlet is formed of flexible materials.

5. The device of claim 1, wherein the outlet is formed of flexible materials.

6. The device of claim 1, wherein the pressure resistance apparatus comprises a deformable diaphragm.

7. The device of claim 6, wherein the deformable diaphragm comprises a flexure threshold corresponding to the threshold value.

8. The device of claim 6, wherein the deformable diaphragm comprises a first side configured to seal the inlet from the outlet when the pressure force is equal to or below the threshold value.

9. The device of claim 6, wherein the deformable diaphragm is configured to deform when the pressure force from the first portion of the eye exceeds the threshold value, wherein deformation of the deformable diaphragm fluidly couples the inlet and the outlet to enable the drainage of the fluids from the first portion of the eye to the second portion of the eye.

10. The device of claim 6, further comprising a reservoir disposed between the inlet and the deformable diaphragm.

11. The device of claim 1, wherein the pressure resistance apparatus comprises an elastic device configured to expand and compress to maintain the IOP.

12. The device of claim 11, wherein the elastic device is configured to expand to seal the inlet from the outlet when the pressure force is equal to or below the threshold value.

13. The device of claim 11, wherein the elastic device is configured to compress to fluidly couple the inlet and the outlet when the pressure force exceeds the threshold value.

14. The device of claim 11, wherein the elastic device is coupled to a plunger configured to be acted upon by the pressure force.

15. The device of claim 11, wherein the elastic device comprises a compression spring.
